(19) United States
(12) Patent Application Publication
(10) Pub. No.: US 2022/0257640 A1
(43) Pub. Date: Aug. 18, 2022

(54) NUTRITIONAL FORMULATION FOR CANCER PREVENTION

(71) Applicant: Hyun Ho Chung, Fullerton, CA (US)

(72) Inventor: Hyun Ho Chung, Fullerton, CA (US)

(21) Appl. No.: 17/669,581

(22) Filed: Feb. 11, 2022

Related U.S. Application Data
(60) Provisional application No. 63/149,152, filed on Feb. 12, 2021.

(51) Int. Cl. A61K 31/737; A61K 31/047; A61K 31/01; A61K 31/12; A61K 31/198; A61K 31/122; A61K 31/375; A61K 36/07; A61K 36/185; A61K 36/9066

(52) U.S. Cl. CPC A61K 31/737; A61K 31/047; A61K 31/01; A61K 31/12; A61K 36/9066; A61K 31/122; A61K 31/375; A61K 36/07; A61K 36/185; A61K 31/198

(57) ABSTRACT

A nutritional supplement containing specific natural ingredients and composition to target cancer vulnerability. The supplement includes between 100 to 300 mg Fucoidan, between 5 and 20 mg Lutein, between 5 and 20 mg Lycopene, between 100 and 300 mg Curcumin, between 5 and 20 mg Astaxantin, between 10 and 100 mg of Siberian Chaga extract powder and between 50 and 200 mg Vitamin C, natural vitamin C from Acerola Cherry, Astaxanthin extract (96%), Curcumin (95%), Lycopene extract (96%), Lutein extract (80%), Siberian Chaga extract, and Fucoidan extract from a brown algae, Undaria pinnatifida. The administration of combinations of these nutrients in set ratio increase anti-cancer activities and the body's ability to prevent cancer. These combinations are also important in helping the body reach the proper immune balance required for maximized function and/or optimal health. All amounts by weight.

### CROSS-REFERENCE TO RELATED APPLICATIONS

The present application claims the priority of U.S. Provisional Patent Application Ser. No. 63/149,152 filed Feb. 12, 2021, which application is incorporated in its entirety herein by reference.

### BACKGROUND OF THE INVENTION

The invention relates to the use of dietary ingredients derived from brown algae, and plants to prevent cancer. This invention particularly relates to the composition of natural ingredients showing the potent inhibitory effect of cancer cell growth and survival.

According to estimates from the International Agency for Research on Cancer (IARC), there were 17.0 million new cancer cases and 9.5 million cancer deaths worldwide in 2018. In 2030, there will be a projected 23.6 million cancer cases around the world. This makes cancer the leading cause of death in the developed world and the second leading cause of death in the developing world.

The increasing cancer burden is due to several factors, including population growth and aging as well as the changing prevalence of certain causes of cancer linked to social and economic development. This is particularly true in rapidly growing economies, where a shift is observed from cancers related to poverty and infections, to cancers associated with lifestyles more typical of industrialized countries.

Effective prevention efforts may explain the observed decrease in incidence rates for some cancers, such as lung cancer (e.g. in men in Northern Europe and North America) and cervical cancer (e.g. in most regions apart from Sub-Saharan Africa). However, new data shows that most countries are still faced with an increase in the absolute number of cases being diagnosed and requiring treatment and care.

According to a National Institutes of Health (NIH) analysis, based on the growth and aging of the U.S. population, medical expenditures for cancer treatment and care in the year 2020 in US are projected to reach at least $158 billion—an increase of 27% from 2010. This number could jump to $207 billion with the development of more expensive treatment options and diagnostic tools.

As a result, World Health Organization (WHO) emphasizes cancer prevention that offers the most cost-effective long-term strategy for the control of cancer. However, millions of Americans do not realize the everyday choices that impact their cancer risk. One in two Americans are now overweight or obese; but only half understand that this places them at increased risk for many cancers.

Prevention is therefore key to both better health and lower health-care costs in the future. IACR estimates that close to 30% of common cancers in the US, nearly 350,000 cases, can be prevented through a healthy diet, maintaining a healthy weight, and getting 30 minutes of physical activity each day. Adding in not smoking and avoiding sun damage, that estimate grows to 50%.

The rising health care costs associated with cancer pose a huge finance burden for the patients, their families, communities, insurance companies and state and federal governments. This highlights the importance for continuing advancing the science of cancer prevention.

In this regard, cancer chemoprevention, the use of natural ingredients to inhibit, retard, or reverse the process of carcinogenesis, is one of the important approaches for easing this challenging public health burden. Recently it has been reported that natural products have played a critical role in cancer chemoprevention studies.

From a historical perspective, the concept of cancer chemoprevention appears to have evolved through epidemiological observations. For example, cancer incidence rates appear to be lower in population groups with diets rich in fruits and vegetables or Mediterranean diet. Interestingly, in many cases, this has led to the isolation and study of many important dietary ingredients, such as fucoidan from brown algae, lycopene from tomatoes, curcumin from turmeric, lutein from marigold, astaxanthine from algae and chaga mushroom extract. Combination regimens of these ingredient have shown the efficacy in cancer chemoprevention activity.

Cancer is driven by genetic and epigenetic alterations that allow cells to overproliferate and escape mechanisms that normally control their survival and migration. Many of these alterations map to signaling pathways that control cell growth and division, cell death, cell fate, and cell motility. These alterations can also cause distortions of wider signaling networks that fuel cancer progression, such as changes in the microenvironment, angiogenesis, and inflammation. Mutations that convert cellular proto-oncogenes to oncogenes can cause hyperactivation of these signaling pathways, whereas inactivation of tumor suppressors eliminates critical negative regulators of signaling. For example, PI3K-Akt and Ras-Erk pathways are typical pathways, in which alterations dysregulating the pathways produce many of the characteristic features of cancer cells. These alterations in cell proliferation and survival signaling pathways are closely associated with cancer dependencies.

The demonstration that cancers are often dependent on specific driver oncogenes has motivated efforts to find and exploit these targets therapeutically. Many potential targets have been identified to be vulnerable in variety of cancers. In comparison to normal cells, cancer cells rely inordinately on pathways that abrogate a variety of cancer related stressors that include DNA damage replication stress, proteotoxic stress, mitotic stress, metabolic stress, and oxidative stress. Even though proteins within these pathways may be essential in all cells, genetic alteration may induce a state where reliance on these pathways creates a therapeutic window as a result of cancer-specific stresses.

### BRIEF SUMMARY OF THE INVENTION

The present invention addresses the above and other needs by providing a nutritional formula containing specific natural ingredients and composition to target cancer vulnerability. It has been discovered that the administration of combinations of these nutrients in set ratio increase anti-cancer activities and the body's ability to prevent cancer. These combinations are also important in helping the body reach the proper immune balance required for maximized function and/or optimal health.

The present invention is directed to a nutritional supplement having fucoidan, lutein, lycopene, curcumin, astaxantin, and vitamin C. The nutritional supplement is adapted to provide nutritional ingredients including between 100 to 300 mg fucoidan, between 5 to 20 mg lutein, between 5 and 20 mg lycopene, between 100 to 300 mg curcumin, between 5 to 20 mg astaxantin, and between 50 to 200 mg vitamin C. The supplement also contains between 10 and 100 mg of siberian chaga extract powder. To enhance the body's ability to absorb these nutritional ingredients, the supplement may also contain alpha cyclodextrin.

In another embodiment, the present invention is directed to a nutritional supplement having microencapsulation of lutein, lycopene, curcumin, vitamin C, and astaxantin wherein the lutein, lycopene, curcumin, vitamin C, and astaxantin are present in a ratio of about 1:1:20:20:1 (mg).

In yet another embodiment, the present invention may include ingredients to meet the special nutritional needs of adults over the age of 50.

### DETAILED DESCRIPTION OF THE INVENTION

The following description is of the best mode presently contemplated for carrying out the invention. This description is not to be taken in a limiting sense, but is made merely for the purpose of describing one or more preferred embodiments of the invention. The scope of the invention should be determined with reference to the claims. Amounts of ingredients are by weight.

One or more of the embodiments below may be combined in order to provide a unitary nutritional supplement containing the nutrients from each embodiment in the amounts specified. Unless indicated otherwise, the combinations below are for nutritional supplements for adults of all ages as well as adults over the age of 50.

### Fucoidan, Lutein, Lycopene, and Curcumin

According to one embodiment of the present invention, the nutritional supplement includes fucoidan, lutein, lycopene, curcumin, and L-arginine. This particular combination of nutrients results in enhanced anti-cancer activities, bioavailability compared to prior art combinations, which are comprised of lutein, lycopene, and curcumin. Preferably, each ingredient in the nutritional supplement is microencapsulated. In other words, the nutritional supplement may include microencapsulated fucoidan, lutein, lycopene, and curcumin. Preferably, the nutritional supplement also includes L-arginine to improve immune function, absorption and bioavailability.

The ratio of fucoidan, lutein, lycopene, and curcumin, is typically about 30:1:1:20 (by weight). This combination of nutrients helps support cancer prevention. Fucoidan is a naturally derived compound from a variety of different species of brown algae showing anticancer properties. In terms of molecular mechanisms of anticancer activities, fucoidan is a multipotent molecule that is able to affect pathways and types of cellular receptors. The main pathways influenced by fucoidan are the PI3K/AKT, the MAPK pathways, and the caspase pathway. PTEN seems to be important in the fucoidan mediated effect on the AKT pathway. Furthermore, the interaction with VEGF, BMP, TGF-beta, and estrogen receptors also seem to be involved in anticancer activities. Particularly, fucoidan as an adjunct seems to have beneficial effects on both the enhanced effectiveness of chemotherapy and reduced toxicity in healthy cells.

Lutein belongs to the xanthophyll family of carotenoids concentrated in leafy green vegetables such as maize and orange pepper. It is known as “the eye vitamin” and is taken by mouth to prevent eye diseases that may lead to vision loss in older adults, referred to as Age-related Macular Degeneration (AMD) and cataracts. In addition to helping eye health, lutein possesses a range of biological properties including anti-inflammatory, anti-oxidant and anti-cancer actions, and has growth inhibitory and cytotoxic effects in several cancer cell lines and animal models. Recent studies have shown that lutein inhibits growth of rat prostate carcinoma cells (AT3 cell) and human prostate cancer cells (PC3), alters mouse mammary tumor development, and induces apoptosis in transformed but not in normal human mammary cells.

Lycopene, an antioxidant carotenoid rich in tomatoes has been reported to be a more stable and potent singlet oxygen quenching agent compared to other carotenoids. In addition to its antioxidant properties, lycopene shows an array of biological effects including cardioprotective, anti-inflammatory, antimutagenic and anticarcinogenic activities. The mechanisms underlying the inhibitory effects of lycopene on carcinogenesis could Involve Reactive Oxygen Species (ROS) scavenging, upregulation of detoxification systems, interference with cell proliferation, induction of gap-junctional communication, inhibition of cell cycle progression and modulation of signal transduction pathways.

Curcumin (diferuloylmethane) is a polyphenol derived from the Curcuma longa plant, commonly known as turmeric. Curcumin has been used extensively in ayurvedic medicine for centuries, as it is nontoxic and has a variety of therapeutic properties including anti-oxidant, analgesics, anti-inflammatory and antiseptic activity. More recently curcumin has been found to possess anti-cancer activities via its effect on a variety of biological pathways involved in mutagenesis, oncogene expression, cell cycle regulation, apoptosis, tumorigenesis and metastasis. Curcumin has shown anti-proliferative effect in multiple cancers, and is an inhibitor of the transcription factor NF-kB and downstream gene products (including c-myc, Bcl-2, COX-2, NOS, Cyclin D1, TNF-alpha, interleukins and MMP-9). In addition, curcumin has an effect on a variety of growth factor receptors and cell adhesion molecules involved in tumor growth, angiogenesis and metastasis.

### Siberian Chaga Extract Powder

Medicinal mushroom have an established history of use in traditional oriental therapy and nutritionally functional foods. Inonotus obliquus (chaga mushroom) belonging to the family Hymenochaetaceae of Basidiomycetes, grows on the trunks of mature live birch trees in cold climates. It has been used as a folk remedy for cancer, digestive system diseases, and various ailments in Russia and other northern European countries. The conk that is used medicinally is comprised of wood from the substrate tree and mycelium of the invasive fungus. In vitro, chaga extract has demonstrated antitumor, anti-mutagenic, antiviral, antiplatelet, antidiabetic, antioxidant, and analgesic effects. In vivo studies also demonstrated immunomodulating, anti-inflammatory, and pain-relieving properties. In animal studies, chaga extract displayed anti-allergic, cognition-enhancing, and antioxidant activities. In murine colitis models, chaga exerted anti-inflammatory effects. Oral administration of polysaccharides from chaga increased exercise endurance and biological measures related to fatigue. Antidiabetic effects have also been observed.

Particularly, chaga extracts and constituents have demonstrated inhibitory and proapoptotic effects against colon, lung, and liver cancer cells. Inotodiol from chaga exerted antitumor effects against cervical cancer cells. In some studies, chaga demonstrated selective apoptosis in tumor cells with no effects on healthy cells. In animal models, it inhibited melanoma cell growth. In summary, the extracts of chaga mushroom has shown potent antitumor activities and have been used to treat cancer worldwide. Continuous intake of aqueous extract from chaga mushroom may help prevent and treat cancer.

### Astaxantin and Vitamin C

Astaxanthin, one of the most powerful antioxidants derived from natural resources, is a xanthophyll carotenoid contained in Haematococcus pluvialis, Chlorella zofingiensis, Chlorococcum, and Phaffia rhodozyma. It accumulates up to 3.8% on the dry weight basis in H. pluvialis. Recent studies have shown that astaxanthin has a great potential biological activity in in vitro and in vivo models. Particularly, astaxanthin, acting as an antioxidant and anticancer agent, prevents various diseases such as diabetes, cardiovascular diseases, and neurodegenerative disorders, cancer, and also stimulates immune response.

Antioxidants, reducing ROS such as superoxide, hydrogen peroxide and hydroxy radical, decrease mutagenesis and carcinogenesis by inhibiting oxidative damage to cells. Astaxanthin, nature's most powerful antioxidant has a significant antitumor activity compared to other carotenoids like beta-carotene. Recent studies revealed that astaxanthin inhibits cell proliferation of various cancer cells. In animal models, astaxanthin inhibited cell death, cell proliferation and mammary tumors in chemically induced male/female rats and mice. H. pluvialis extract also inhibits the growth of human colon cancer cells by arresting cell cycle progression and promoting apoptosis. These results support the assumption that protecting body tissues from oxidative damage with daily ingestion of natural astaxanthin may be a natural, practical and beneficial strategy in cancer prevention and health care management.

Vitamin C is considered to be one of the most prevalent antioxidative components of fruit and vegetables. It has chemo-preventive effects without apparent toxicity at doses higher than 60 mg/day. It has also been used as a dietary supplement intended to prevent oxidative stress-mediated chronic diseases such as cancer, cardiovascular diseases, hypertension, stroke, and neurodegenerative disorder. Currently, the US Department of Agriculture and the National Cancer Institute recommend the consumption of a minimum of 5 servings of fruit and vegetables/day to prevent cancer. Therefore, should these recommendations be followed, a daily consumption of 200-280 mg vitamin C might be achieved. Recent studies have shown that vitamin C protects cells from oxidative DNA damage, thereby blocking the initiation of carcinogenesis, and the chemo-preventive effects of vitamin C may be linked to the inhibition of tumor promotion as well as to the blocking of tumor initiation. These results strongly support the potential chemo-preventive effects of vitamin C in carcinogenesis.

Another embodiment of the present invention thereby is a nutritional supplement containing lutein, lycopene, astaxanthin, curcumin, and vitamin C (registered as a trademark by LLACC). Preferably, the nutritional supplement included microspheres or microencapsulation containing either separately or together, the lutein, lycopene, astaxanthin, curcumin, and vitamin C. The ratio of lutein, lycopene, astaxanthin, curcumin, and vitamin C is typically about 1:1:1:20:10. This combination of antioxidants helps support the natural cell repair process and prevents cell damage resulting in chronic diseases of oxidative origins, such as cancer, cardiovascular diseases, diabetes, stroke, neurodegenerative disorders, age-related macular degeneration and cataracts. Furthermore, the specific ratio of theses nutritional ingredients prevents them from competing with one another in order to maximize absorption and bioavailability.

Preferably, the nutritional supplement includes about 10 mg lutein, about 10 mg lycopene, about 10 mg astaxanthin, about 200 mg curcumin, and about 200 mg vitamin C per serving. More preferred embodiment of the nutritional supplement includes about 300 mg fucoidan extract and 50 mg of Siberian chaga extract.

### Definitions

As used herein, the term “about” means within 10% of a given value, preferably within 5%, and more preferably within 1% of a given value. Alternatively, the term “about” means that a value can fall within a scientifically acceptable error range for that type of value, which will depend on how qualitative a measurement can be given the available tools.

The term “fucoidan” refers to natural sulfated polysaccharide found in the cell wall of many species of brown seaweed. Recent studies have shown that it has antitumor, antiangiogenic, antiviral, antiarthritic, and immunomodulatory effects. Fucoidan also exhibits neuroprotective, radioprotective, and antiulcer properties.

The term “chaga mushroom extract” refers to the extract from a natural chaga mushroom that is found on birch and other trees in cold climates. It has been used as a folk remedy for cancer, digestive system diseases, and various ailment in Russia and other northern European countries. The conk that is used medicinally is comprised of wood from the substrate tree and mycelium of the invasive fungus. Recent studies have shown that it has antitumor, anti-mutagenic, antiviral, antiplatelet, antidiabetic, antioxidant, immunomodulating, anti-inflammatory, pain-relieving, and analgesic effects. Chaga also exhibits anti-allergic, and cognitive-enhancing properties.

The term “lutein” refers to a natural carotenoid in the subfamily xanthophylls, also known as oxy-carotenoids. It is a phytochemical found in dark green vegetables, flowers in marigold and fruits such as kale, spinach, collard greens, carrots, and avocado pears. Lutein has been used for a dietary supplement as an antioxidant and shows a range of biological properties including anti-inflammatory, anti-oxidant and anti-cancer actions. It also has shown growth inhibitory and cytotoxic effects in several cancer cell lines and animal models.

The term “lycopene” refers to natural antioxidant carotenoid rich in tomatoes, which has been reported to be a more stable and potent singlet oxygen quenching agent compared to other carotenoids. In addition to its antioxidant properties, lycopene shows an array of biological effects including cardioprotective, anti-inflammatory, antimutagenic and anticarcinogenic activities.

The term “astaxanthin” refers to a natural antioxidant that belongs to a group of chemicals called carotenoids. It occurs naturally in certain algae and causes the pink or red color in salmon, trout, lobster, shrimp, and other seafood. Recent studies have shown that it can be taken by mouth for treating Alzheimer's diseases, Parkinson's diseases, stroke, high cholesterol, liver diseases, age-related macular degeneration (age-related vison loss), dyspepsia, rheumatoid arthritis, and preventing cancer. It is also used to treat metabolic syndrome, which is a group of conditions that increase the risk of heart diseases, stroke, and diabetes.

The term “curcumin” refers to a natural polyphenol derived from the Curcuma longa plant, commonly known as turmeric. Curcumin has been used extensively in Ayurvedic medicine for centuries, as it is nontoxic and has a variety of therapeutic properties including anti-oxidant, analgesics, anti-inflammatory and antiseptic activity. More recently curcumin has been found to possess anti-cancer activities via its effect on a variety of biological pathways involved in mutagenesis, oncogene expression, cell cycle regulation, apoptosis, tumorigenesis and metastasis.

The term “Vitamin C” refers to natural ascorbic acid that is one of the safest and most effective antioxidants. Recent studies have shown that the benefits of vitamin C include protection against immune system deficiencies, cardiovascular diseases, prenatal health problems, eye diseases, and even skin wrinkling. It also has protective effects against cancer, stroke, and other chronic diseases by improving immune response.

### Formulations

The nutritional supplement is preferably a solid dosage form, such as capsule or tablet, which suitable for once, or twice daily administration. Preferably, the nutritional supplement is a capsule form.

The nutritional supplement may be composed one or more excipients. Suitable excipients include, but are not limited to, alpha-cyclodextrin, silicon dioxide, magnesium stearate, microcrystalline cellulose, and preservatives.

The nutritional supplement of the present invention may be prepared by methods known in the art. The following examples illustrate the invention without limitation. All parts are given by weight unless otherwise indicated.

### Example 1

The formulation of a nutritional supplement intended for regular use by a woman older than 50 years of age is shown below. (2 capsules/serving)

| Ingredients (per Serving) | Unit of Measure | Amount |
|---|---|---|
| Fucoidan Extract | mg | 300 |
| Siberian Chaga Extract Powder | mg | 50 |
| Lutein (Extract, 80% Lutein) | mg | 10 |
| Lycopene (Extract, 96% Lycopene) | mg | 10 |
| Turmeric Extract (Curcumin 95%) | mg | 200 |
| Astaxanthin (Extract, 96% | mg | 10 |
| Vitamin C (Acelora Cherry) | mg | 100 |

The formulation of a nutritional supplement intended for regular use by a man older than 50 years of age is shown below (2 capsules/serving)

| Ingredients (per Serving) | Unit of Measure | Amount |
|---|---|---|
| Fucoidan Extract | mg | 300 |
| Siberian Chaga Extract Powder | mg | 50 |
| Lutein (Extract, 80% Lutein) | mg | 10 |
| Lycopene (Extract, 96% Lycopene) | mg | 10 |
| Turmeric Extract (Curcumin %) | mg | 200 |
| Astaxanthin (Extract, 96%) | mg | 10 |
| Vitamin C (Acerola Cherry) | mg | 100 |
| L-Arginine | mg | 300 |

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, articles, publications and test methods mentioned above are hereby incorporated by reference in their entirely.

While the invention herein disclosed has been described by means of specific embodiments and applications thereof, numerous modifications and variations could be made thereto by those skilled in the art without departing from the scope of the invention set forth in the claims.

I claim:

1. A nutritional supplement adapted to provide nutritional elements, comprising:
fucoidan;
lutein; and
lycopene.

2. The nutritional supplement of claim 1, further including curcumin.

3. The nutritional supplement of claim 2, wherein the ratio of fucoidan, lutein, lycopene, and curcumin, is about 30:1:1:20 (by weight).

4. The nutritional supplement of claim 1, further including L-arginine.

5. The nutritional supplement of claim 1, further including siberian chaga extract powder.

6. The nutritional supplement of claim 1, further including astaxantin and vitamin c.

7. The nutritional supplement of claim 1, further including:
curcumin;
astaxantin;
siberian chaga extract powder; and
vitamin c,
and comprising:
between 100 to 300 mg fucoidan;
between 5 and 20 mg lutein;
between 5 and 20 mg lycopene;
between 100 and 300 mg curcumin;
between 5 and 20 mg astaxantin;
between 10 and 100 mg of siberian chaga extract powder; and
between 50 and 200 mg vitamin c,
all ingredients by weight.

8. The nutritional supplement of claim 1, wherein the fucoidan comprises fucoidan extract from a brown algae,
further including:
natural vitamin c from acerola cherry;
astaxanthin extract;
curcumin;
siberian chaga extract; and
Undaria pinnatifida; and
comprising:
astaxanthin extract (96%);
curcumin (95%);
lycopene extract (96%);
lutein extract (80%),
all amounts by weight.

9. The nutritional supplement of claim 1, further comprising:
about 300 mg Fucoidan extract;
about 50 mg Siberian Chaga extract;
about 10 mg Lutein extract (80%) about 10 mg Lycopene extract (96%);
about 200 mg Curcumin extract (95%);
about 10 mg Astaxanthin extract (96%);
natural vitamin C from Acerola; and
L-Arginine,
all amounts by weight.

10. The nutritional supplement of claim 9, further including about 300 mg L-Arginine by weight.

11. A nutritional supplement adapted to provide nutritional elements comprising:
lutein;
lycopene;
astaxanthin; and
vitamin c,

12. The nutritional supplement of claim 11, further including curcumin, wherein the ratio of lutein, lycopene, astaxanthin, curcumin, and vitamin c is about 1:1:1:20:10, all amounts by weight.

13. The nutritional supplement of claim 12, comprising:
about 10 mg lutein;
about 10 mg lycopene;
about 10 mg astaxanthin;
about 200 mg curcumin; and
about 200 mg vitamin C per serving.

14. The nutritional supplement of claim 13, further including:
about 300 mg fucoidan extract; and
50 mg of Siberian chaga extract.

15. The nutritional supplement of claim 11 taken 2 capsules a serving, further including:
fucoidan extract;
siberian chaga extract powder; and
turmeric extract, and comprising:
about 300 mg fucoidan extract;
about 50 mg siberian chaga extract powder;
about 10 mg of lutein (extract, 80% lutein);
about 10 mg lycopene (extract, 96% lycopene);
about 200 mg turmeric extract (curcumin 95%);
About 10 mg astaxanthin (extract, 96%); and
about 100 mg vitamin C (acelora cherry).

16. The nutritional supplement of claim 11 taken 2 capsules a serving, further including:
fucoidan extract;
siberian chaga extract powder;
turmeric extract, and
L-Arginin; and
comprising:
about 300 mg fucoidan extract;
about 50 mg siberian chaga extract powder;
about 10 mg of lutein (extract, 80% lutein);
about 10 mg lycopene (extract, 96% lycopene);
about 200 mg turmeric extract (curcumin 95%);
About 10 mg astaxanthin (extract, 96%);
about 100 mg vitamin C (acelora cherry); and
about 300 mg L-Arginin.

17. A nutritional supplement adapted to provide nutritional elements comprising a microencapsulation of:
fucoidan;
lutein;
lycopene; and
curcumin.

18. The supplement of claim 17, wherein the ratio of fucoidan, lutein, lycopene, and curcumin, is about 30:1:1:20 (by weight).

19. The supplement of claim 17, wherein the ratio of fucoidan, lutein, lycopene, and curcumin, is about 30:1:1:20 (by weight).

20. The supplement of claim 17, further including L-arginine.
vitamin C; and
astaxantin,
wherein the lutein, lycopene, curcumin, vitamin C, and astaxantin are present in a ratio of about 1:1:20:20:1 by weight.
